(19) United States
(12) Patent Application Publication
(10) Pub. No.: US 2023/0377137 A1
(43) Pub. Date: Nov. 23, 2023

(54) ANALYSIS METHOD

(71) Applicants: QBD (QS-IP) Limited, St Helier (JE); Quotient Suisse SA, Eysins (CH)

(72) Inventors: Pascal RADENEZ, St Helier (JE); Emmet MCGAURAN, St Helier (JE)

(21) Appl. No.: 18/030,036

(22) PCT Filed: Oct. 8, 2021
(86) PCT No.: PCT/EP2021/077938

§ 371 Date: Apr. 3, 2023

(30) Foreign Application Priority Data
Oct. 9, 2020 (GB) 2016063.6

(51) Int. Cl. G06T 7/00; G01N 33/52

(52) U.S. Cl. CPC G06T 7/0012; G01N 33/528; G06T 2207/20021

(57) ABSTRACT

A computer implemented method of analysing assays performed at respective assay sites of an array or microarray that comprises a plurality of assay sites, the method comprising receiving at least one image of the assay sites of the array or microarray; for each of the assay sites, processing the at least one image to determine at least one metric representative of the degree of reaction at that assay site; for each of the assay sites, determining one or more parameters for that assay site, wherein the one or more parameters for at least one of the assay sites of the array or microarray are different from the one or more parameters for at least one other of the assay sites of the array or microarray; and for each of the assay sites, determining an extent of the reaction at that assay site from the at least one metric for that assay site and the one or more parameters for that assay site. Also disclosed is a correspondingly configured computerized analysis system.

### FIELD

The present disclosure relates to analysis systems and methods for analysing reactions, such as analysis systems and methods for analysing assays.

### BACKGROUND

Analysis systems are available for automating the analysis of chemical assays. Such assays are generally performed in a regular array of print areas that are configured to hold reactants and a test sample. Such assays generally test for the presence or level of an analyte in the test sample. A response, often in the form of a change in degree of opacity, colour, size or other detectable change, is associated with the presence or level of the analyte. The analysis system is provided with a sensor, typically a digital camera, for identifying the response in each print in the array. In this way, for example, the digital camera image can be reviewed and the response (e.g. degree of change in colour or opacity) can be identified in order to determine the presence or level of analyte in any given print area.

Typically, the same type of assay is performed in each print area but it can be desirable to perform different types of assay in different print areas of the array.

Manual review of the response shown in the digital camera image can be performed, which allows a degree of expertise and human judgement to be applied, but is often slow, labour intensive and prone to human error. Automated determination of the response can be much faster and have a high throughput, but can sometimes be associated with errors, particularly if there are unforeseen or unaccounted for effects or if the response is marginal or borderline, commonly referred to as edge cases.

At least some examples of the present disclosure seek to improve the automated analysis of assays performed on arrays or microarrays comprising a plurality of print areas, particularly where different assays are performed in different print areas.

### SUMMARY

Various aspects of the present invention are defined in the independent claims. Some preferred features are defined in the dependent claims.

According to a first example of the present disclosure is a computer implemented method of analysing a plurality of reactions performed at respective sites in an array, that comprises a plurality of the sites, the method comprising:
receiving at least one image of the plurality of sites of the array;
for each of the plurality of sites, processing the at least one image to determine at least one image metric representative of the degree or extent of reaction at that site;
for each of the plurality of sites, determining one or more parameters for that site, wherein the parameters for at least one of the sites are different from the parameters for at least one other of the sites; and
for each of the plurality of sites, determining an extent of the reaction from the at least one image metric for that site and the one or more parameters for that site.

The array may be or comprise or be comprised in a microarray, e.g. a multiplexed microarray, or a hybridized array.

The reactions may be or comprise assays. The sites of the array may be or comprise assay sites, wells, print areas or reaction sites of the array. The reaction may comprise a chemical and/or biological reaction. The assay may comprise one or more reagents at the site. The assay may comprise addition of a sample to the reagents. The presence of an analyte in the sample may cause the reaction with the reagents. The reaction may result in a spot at the site, the spot resulting from and being indicative of a measurable change caused by the reaction. At least one property of the printed material may result in the reaction creating the spot characterised by change of one or more of: opacity, intensity, colour, size, and/or the like, which may be indicative of the degree or extent of reaction. The one or more criteria and/or parameters for a site may be, comprise or be representative of criteria and/or parameters of the spot or the part of the image representing the spot. The extent of reaction may comprise or be indicative of a spot grade. The determining of an extent of reaction may be, be comprised in or may comprise a grading or a determination of spot grade. The extent of reaction or spot grade may be qualitative, e.g. reacted or not, or quantitative, e.g. a value for extent or degree of reaction. The at least one metric for the site may be or comprise a metric of the spot.

The at least one image may comprise one or more images of all of the plurality of assay sites or a collection of images that individually may cover one or some of the plurality of assay sites but collectively cover all of the plurality of assay sites.

The criteria and/or parameters may comprise at least one criterion with an associated threshold or range. At least one criterion with an appropriate threshold or range may be indicative of the extent of reaction or spot grade. The extent of reaction or spot grade may comprise one or more of: a reaction, no reaction, or one or more values representing extent of reaction and/or an indication that the extent of reaction is indeterminate. The indication that the extent of reaction is indeterminate for a site may be or comprise a determination that the at least one image metric determined for that site is between a value or range of values indicative of no reaction and a value or range of values indicative of a reaction. The determining of the extent of reaction for a site may comprise comparing the at least one metric for that site to a reference function, or one or more values, comprising or dependent on the criteria and/or parameters for that site. The determining of the extent of the reaction for the site may comprise determining whether or not the at least one metric for that site is above or below the at least one threshold or within or out with the at least one range or does or doesn't meet at least one of the criteria, which may thereby determine the extent or degree of reaction or spot grade.

The criteria and/or parameters for a site may depend on one or more of: an assay type performed at the site, a location of the site on the array, a sample type at the site and/or the like.

The criteria and/or parameters for a site may be unique or at least tailored, adapted or selected for that site. The criteria and/or parameters may be pre-determined, e.g. determined before the analysing of the plurality of reactions. The criteria or parameters may be for and/or specific to the assay type performed at the site, the location of the site on the array, the sample type at the site and/or the like. The method may comprise, for at least one or each site, determining or identifying the assay type for the assay performed at the site, the location of the site on the array, the sample type at the site and, for the respective site, selectively applying the criteria or parameters for the determined assay type, location of the site on the array, and/or sample type.

The criteria and/or parameters for a site may be fixed. The criteria and/or parameters for each given assay type, location of the site on the array, and/or sample type may be fixed. The criteria and/or parameters for a site may be selectable from amongst a plurality of predetermined criteria and/or parameters associated with different assay type, location of the site on the array, and/or sample type, but may be fixed once selected. The selection of the criteria and/or parameters for at least one or each respective site may comprise selecting the predetermined parameters and criteria and/or parameters for the assay type, location of the site on the array, and/or sample type of that respective site. The plurality of sites (e.g. the assay sites or print areas) may be comprised in an array of sites. The sites may be arranged in rows. Each row of sites may be staggered with respect to at least one or each adjacent row of sites. The array may comprise alternating staggered rows of sites, where each alternate row of sites may be aligned. The array may accord to American National Standards Institute (ANSI)/Society for Laboratory Automation and Screening (SLAS) standards. The array may comprise one or more control sites, which may be located at one or more or each outer corner of the array or of the array of sites. The control sites may be arranged at the apexes of a parallelogram.

The at least one metric for a site may comprise or be dependent on or representative of pixel intensities for an area of the image representing at least part of that site, e.g. the spot at that site. The at least one metric for a site may comprise or be dependent on or representative of a mean value of the pixel intensities for at least part of the area of the image representing that site, e.g. the spot at that site. The at least one metric for a site may comprise or be dependent on or representative of a difference between pixel intensities or a mean value of the pixel intensities for at least part of an area of the image representing the spot at that site, and background pixel intensities or a mean value of the background pixel intensities, e.g. for part of the image representing an area that is not a spot. The at least one metric for a site may comprise or be dependent on or representative of a measure of spread or standard deviation of background pixel intensities.

The at least one metric for a site may be or comprise signal to standard deviation ratio (SSR), e.g. the difference between the mean value of the pixel intensities for at least part of the area of the image representing the spot at that site and the mean value of the pixel intensities for at least part of the background, all divided by the measure of spread or standard deviation of the background pixel intensities for at least the part of the background.

The at least one metric for a site may comprise or be dependent on or representative of a measure of spread or standard deviation of the pixel intensities or a mean value of the pixel intensities for at least part of the area of the image representing the spot at that site. The at least one metric for a site may comprise or be dependent on or representative of a percentage or fraction of pixels representative of the spot at that site that are at or above a pixel intensity threshold.

The method may comprise at least one of: cropping the image around the array of sites. The method may comprise gridding the image into segments wherein each segment encloses a spot and/or site.

One or more or each of: the cropping, the gridding and/or the determining of the at least one metric of the spot may comprise using associated cropping, gridding and/or metric determination parameters, which may be common parameters that are shared with one or more or each or every other spot or site with the same assay type, e.g. the common parameters are common to an assay type. The criteria and/or parameters used for determining the extent of the reaction for a site from the at least one image metric for that site may be unique for that site.

The method may comprise pre-processing or cleaning the image, e.g. by filtering or smoothing the image. The filtering or smoothing may be configured to selectively filter out noise from the image.

The cropping of the at least one image may comprise determining a cropped area of the image, which may be a subset of a total area of the image, in which the plurality of sites are shown. The cropping of the image may comprise cropping the image around the plurality of sites on the array. The method may comprise identifying one or more or each control site, which may define outer corners of the array of sites on the array and may define or be used to define the corners of the cropped area of the image.

The method may comprise detecting or identifying the parts of the images corresponding to the spots in the reaction sites. The method may comprise detecting or identifying shapes in the cropped image that fit a description of a spot, e.g. using object recognition techniques. The detecting or identifying the parts of the images corresponding to the spots in the reaction sites may be based on one or more of the parameters, e.g. the assay specific or site specific parameters.

The method may comprise filtering the determined or identified spots in the image. The method may comprise filtering out determined or identified spots for which a value of at least one of the metrics is below a threshold, which may be a pre-determined or determined threshold, and may be comprised in or be derived from at least one of the parameters, e.g. the assay specific or site specific parameters.

The method may comprise identifying the location of reference points, which may be the control sites. The reference points, e.g. the control sites, may be identified by identifying the extreme or most distant sites shown in the image and optionally applying known or predicted geometry of the array. The method may comprise filtering out determined or identified spots using spatial thresholds based on the know or predicted geometry of the array, e.g. a known or predicted arrangement of the sites on the array, which may comprise an arrangement relative to the reference points, such as the control sites. The method may comprise filtering out those spots that are not within an area defined by the control sites at each corner, e.g. outside an area in which the sites are expected to be found.

The method may comprise gridding the image, which may comprise determining a grid comprising a plurality of segments, which may be based on the determined position of the reference points, e.g. control sites, and/or the determined positions of other sites or spots and/or the expected geometry of the sites on the array. Each segment may contain a single corresponding assay site of the array. The cells may be non-overlapping. The cells may correspond with the arrangement of sites on the array. The gridding may be based on one or more of the parameters, e.g. the assay specific or site specific parameters.

The determination of the at least one metric of the sites may comprise determining at least one of the metrics directly from the image. The determination of the at least one metric of the reaction sites may comprise determining at least one metric indirectly from at least one of the metrics determined directly from the image. The determination of the at least one metric of the sites may be based on one or more of the parameters, e.g. the assay specific or site specific parameters.

Non-exhaustive examples of metrics include: mean image background pixel value or intensity, mean standard deviation or spread of image background values or intensities, the mean and/or standard deviations or spreads of pixel values or intensities of a spot, circle radius for a spot, shape radius and/or isoperimetric co-efficient of a spot, edge sharpness or spread of a spot, circle signal to standard deviation ratio, circle mean, circle standard deviation, circle background mean and/or standard deviation or spread, circle intensity value above background, mean, standard deviation and/or spread of image background pixel value or intensity for a spot, a signal to standard deviation ratio (SSR) for a spot, a difference between pixel value or intensity of at least part of a spot and that of the background, and/or the like.

The determining of the one or more parameters for the sites may comprise determining or identifying respective parameters associated with the assay being performed in the respective site and/or associated with the particular site, and applying those parameters for that respective site. The parameters may be predetermined for a given array, assay type, sample type, location, location in the array, site, and/or the like.

In one example, the parameters for the sites may be retrieved from an input file, input by a user using a user input device such as a keyboard, and/or the like.

The method may comprise identifying or receiving one or more identifiers indicating the array or at least one or each of the sites on the array or the assay or assay type being performed in at least one or each site on the array. The determining of the one or more parameters for the sites may comprise looking up or determining the parameters from a look-up table, database, data store, or function that associates parameters with arrays, assay types, sample types and/or sites, e.g. based on the determined identifiers. The one or more indicators may be received from an input device and/or from a data store, e.g. as part of a configuration file. The input device may comprise a user input device such as a keyboard for receiving user input, a machine readable data device such as a barcode or QR code or other machine readable code scanner, a RFID tag reader, an optical tag reader, and/or the like. The one or more indicators may be received from a laboratory information management system (LIMS). The method may comprise reading or otherwise receiving the identifier and may comprise then using the identifier to access the one or more criteria and/or parameters associated with the identifier, e.g. from the data store.

The parameters may be based on past assays, historical data, test assays, modelling or predicted data and/or the like. The determining of the one or more parameters for the sites may comprise determining parameters for one or more or each different assay type and/or site using machine learning, artificial intelligence or some other learning or trained algorithm or function and/or using historical or modelled data and/or the like.

The parameters may comprise global parameters. The global parameters may be parameters that are a function of the assay, i.e. global parameters may vary from assay to assay and/or be associated with a corresponding assay type. The same values for global parameters may be applied to multiple sites or spots. One or more of the gridding of the image, cropping of the image and/or determining of the one or more metric representative of the extent or degree of reaction at each of the sites may be performed based on global parameters.

The parameters may comprise unique parameters. Unique parameters may be parameters that are a function of the particular site on the array or spot, e.g. a function of the assay type and site on the array or spot. The values of unique criteria may be individually variable on a site-by-site basis. At least one or more or all of the parameters used to determine the extent of the reaction at respective assay sites from the at least one image metric for that assay site may be unique parameters.

Non-exhaustive examples of parameters, which may be global criteria, include: a value of maximum image background mean intensity, a value of maximum standard deviation or spread of image background intensity, and/or the like.

The parameters for a site on the array may specify which values of the at least one metric are indicative of a reaction or no reaction at that site on the array. The parameters may specify one or more thresholds, criteria and/or ranges that specify the extent or degree of reaction, e.g. whether the spot is indicative of a reaction, no reaction or is indeterminate.

Non-exhaustive examples of parameters include one or more or each of: a value of a maximum mean intensity for a reaction site, maximum standard deviation or spread of intensity for a reaction site, a limit of signal to standard deviation ratio for a reaction site, a threshold for pixel value or intensity over background for a reaction site to be considered empty or filled, maximum and/or minimum values of circle or shape radius for a detected shape to be considered a spot, a minimum acceptable value of spot edge metric, maximum spot acceptable background cv value to validate the SSR, spot reactive SSR value interval, spot non-reactive SSR value interval, spot reactive mean value interval, spot non-reactive mean value interval, maximum circle acceptable background cv value to validate the SSR, circle reactive SSR value interval, circle non-reactive SSR value interval, circle reactive delta value interval, circle non-reactive delta value interval, and/or the like.

The determining of the extent of the reaction at the assay site or spot may comprise applying one or more of the metrics for that reaction site or spot and the parameters for that reaction site or spot to one or more logic tests, where the result of the one or more logic tests is an indication of whether there has been a reaction at that site or not, an extent or degree of reaction at that site, activity at that site and/or an extent or degree of activity at that site, or presence of an analyte at that site or not. Determining activity at that site and/or an extent of activity at that site may comprise determining if there is something detectable, e.g. a blob, shape of non-background result, but it being unclear whether the activity detected is a spot or reaction or not. The logic test may comprise a comparison of the one or more metrics for the site to the one or more parameters for the site in order to determine the extent of the reaction.

According to a second example of the present disclosure is a processing system configured to implement the method of the first example to analyse one or more assays performed at respective assay sites from a plurality of assay sites of an array.

The processing system may be configured to receive at least one image of the assay sites of the array. The processing system may be configured to, for each of the assay sites, process the at least one image to determine at least one metric representative of the extent or degree of reaction at that assay site. The processing system may be configured to, for each of the assay sites, determine one or more parameters for that assay site, wherein the parameters for at least one of the assay sites of the array are different from the parameters for at least one other of the assay sites of the array. The processing system may be configured to, for each of the assay sites, determine an extent of the reaction at that assay site from the at least one metric for that assay site and the one or more parameters for that assay site.

The processing system may comprise data storage. The processing system may comprise a communications module. The processing system may comprise one or more output devices. The processing system may comprise one or more input devices.

The processing system may comprise one or more processors, which may be single or multi-core processors. The one or more processors may comprise one or more central processing units, graphics processing units, maths co-processors, tensor processing units, and/or the like. The data storage may comprise solid state memory, magnetic memory, optical memory, and/or the like. The communications unit may be configured to communicate via wired and/or wireless communications and may be configured to communicate with remote and/or local systems, e.g. via a network such as a LAN, a WAN, the internet, one or more cellular networks, an Ethernet network, an fibre optic network, and/or the like. The at least one output device may comprise a display or other visual output device, an audio output device, and/or a haptic output device.

The at least on input device may comprise a keyboard, a touchscreen, a trackball, a touchpad, a joystick, a speech recognition based input device, and/or the like. The input device may comprise a machine readable data device such as a barcode or QR code or other machine readable code scanner, a RFID tag reader, an optical tag reader, and/or the like. The processing system may be configured to read or otherwise receive a code associated with an array or site on the array and may comprise then using the code to access the one or more criteria and/or parameters associated with the code, e.g. from the data store.

The processing system may be configured to look up or determine the parameters from a configuration file, look-up table, database, data store, or function that associates respective parameters with respective assay types, sample types and/or sites. The processing system may be configured to communicate with at least one remote processing system. The remote processing system may comprise a server, a cloud computing resource, a workstation, a personal computer, and/or the like. The remote processing system may comprise remote data storage.

According to a third example of the present disclosure is an analysis system for analysing one or more assays performed at respective assay sites from a plurality of assay sites of an array, the analysis system comprising the processing system of the second example described above. The analysis system may comprise at least one imaging device configured or configurable to collect at least one image of the array and communicate the at least one image to the processing system.

The processing system may be configured to receive at least one image of the assay sites of the array. The processing system may be configured to, for each of the assay sites, process the at least one image to determine at least one metric representative of the extent or degree of reaction at that assay site. The processing system may be configured to, for each of the assay sites, determine one or more parameters for that assay site, wherein the parameters for at least one of the assay sites of the array are different from the parameters for at least one other of the assay sites of the array. The processing system may be configured to, for each of the assay sites, determine an extent of the reaction at that assay site from the at least one metric for that assay site and the one or more parameters for that assay site.

According to a fourth example of the present disclosure is a computer program product comprising instructions that, when implemented on a processing system, cause the processing system to perform the method of the first example of the present disclosure. The computer program product may be embodied on a non-transient and/or tangible computer readable medium.

The individual features and/or combinations of features defined above in accordance with any aspect, example or embodiment of the present disclosure or below in relation to any specific embodiment of the disclosure may be utilised, either separately and individually, alone or in combination with any other defined feature, in any other aspect, example or embodiment of the disclosure.

Furthermore, the present disclosure is intended to cover apparatus configured to perform any feature described herein in relation to a method and/or a method of using or producing, using or manufacturing any apparatus feature described herein.

### BRIEF DESCRIPTION OF THE DRAWINGS

These and other aspects of the present disclosure will now be described, by way of example only, with reference to the accompanying Figures, in which:

FIG. 1: a schematic of a computerized assay analysis system;

FIG. 2: an example of an annotated image of assays on an array collected using the system of FIG. 1;

FIGS. 3 to 7: charts showing the variation of assay attributes for a specific metric used in a spot grading process;

FIG. 8: an overview of an assay analysis performed by a computerized assay analysis system, such as that of FIG. 1;

FIG. 9A: an overview of an image analysis that forms part of the assay analysis shown in FIG. 8;

FIG. 9B: a detailed flowchart of the image analysis of FIG. 9A;

FIG. 10: another flowchart of the image analysis of FIGS. 9A and 9B;

FIG. 11: a schematic of a grid matching process performed as part of a cropping process;

FIG. 12: a schematic of a grid for a micro-array produced by the gridding process of FIG. 11;

FIG. 13: a flowchart of a process of determining metrics that forms part of the image analysis of FIGS. 9A, 9B and 10;

FIG. 14: a flowchart of a segmentation process performed as part of the metric determination shown in FIG. 17;

FIG. 15: a flowchart of operation of a grading rule engine for use in the assay analysis of FIG. 8;

FIG. 16: a flowchart illustrating logic for determining suitability for analysis for use in the grading rule engine illustrated with respect to FIG. 15;

FIG. 17: a flowchart illustrating spot determination logic for use in the grading rule engine illustrated with respect to FIG. 15;

FIG. 18: a flowchart illustrating object detection logic for use in the grading rule engine illustrated with respect to FIG. 15;

FIG. 19: a flowchart illustrating bright object determination logic for use in the grading rule engine illustrated with respect to FIG. 15;

FIG. 20: a flowchart illustrating spot classification logic for use in the grading rule engine illustrated with respect to FIG. 15; and

FIG. 21: a flowchart illustrating spot classification confirmation logic for use in the grading rule engine illustrated with respect to FIG. 15.

### DETAILED DESCRIPTION OF THE DRAWINGS

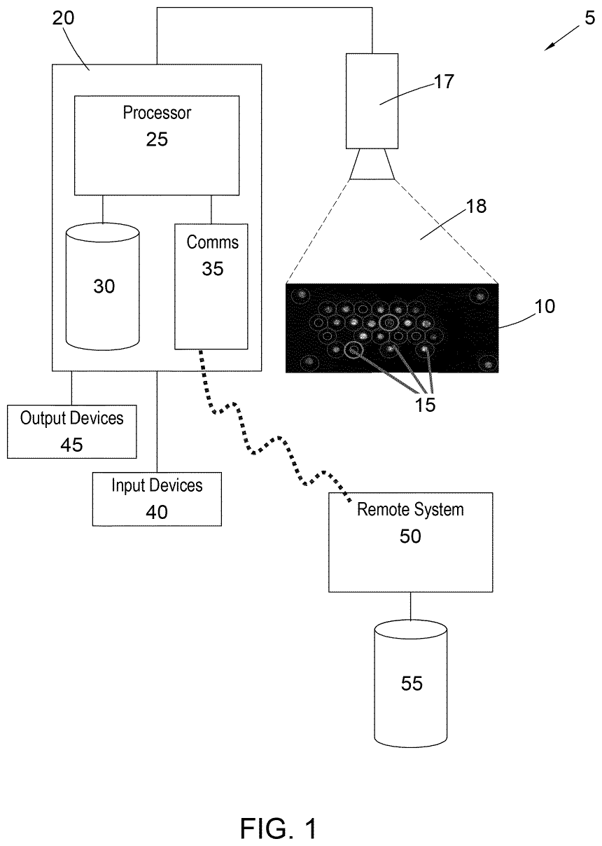

FIG. 1 shows an assay analysis system 5 for analysing assays carried out in an array 10, such as a multiplexed micro-array or hybridized array, that comprises a plurality of assay sites or print areas 15, wherein an individual assay can be performed in each assay site 15 or print area. The system 5 comprises one or more sensors, in this example in the form of a digital camera 17, configured to collect images of the array 10. Although only one camera 17 is shown in FIG. 1, more than one camera 17 could be provided. The camera 17 in this example is configured to capture images of the whole of the array 10, e.g. the field of view 18 of the camera 17 encompasses the whole of the array 10, or at least all of the assay sites 15 of the array 10. However, the camera 17 could be configured to capture images of only some of the assay sites 15 of the array 10, or different cameras 17 could be configured to capture images of different subsets of assay sites 15 of the array 10 or to capture the array 10 from different angles. The system 5 comprises an analysis system 20 that receives the images collected by the camera 10 and is configured to analyse the images to determine states of reaction in specific assay sites 15.

The analysis system 20 comprises a processing system 25, data storage 30, a communications module 35, one or more output devices 45 and one or more user input devices 40. The processing system 25 comprises one or more processors, which could be single core or multi-core processors. The one or more processors include one or more central processing units, and optionally also one or more graphics processing units, maths co-processors, tensor processing units, and/or the like. The data storage 30 could comprise solid state memory, magnetic memory, optical memory, and/or the like. The communications unit 35 can be configured to communicate via wired and/or wireless communications. In this example, the communications unit 35 is configured to communicate with remote and/or local systems, e.g. via a network such as a LAN, a WAN, the internet, one or more cellular networks, an Ethernet network, an fibre optic network, and/or the like. The at least one output device 45 could comprise a display or other visual output device, an audio output device, and/or a haptic output device. The at least on input device 40 could comprise one or more of: a keyboard, a touchscreen, a trackball, a touchpad, a joystick, a speech recognition based input device, an RFID tag reader, a barcode or QR code reader, and/or the like.

The analysis system 20 may be configured to communicate with at least one remote processing system 50. The remote processing system 50 could comprise a server, a cloud computing resource, a workstation, a personal computer, and/or the like. The remote processing system 50 comprises remote data storage 55. In this way, any or all of the method steps described herein, particularly any relating to data processing and/or data storage, could be performed using the processing system 25, the at least one remote processing system 50 or the method steps described herein may be distributed between the processing system 25 and the at least one remote processing system 50.

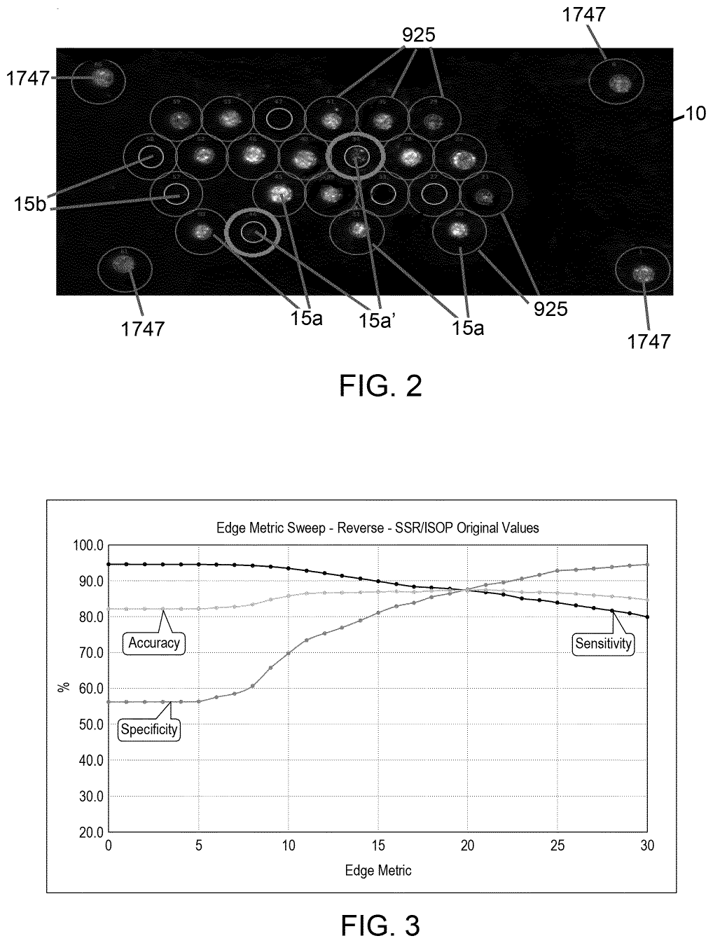

An example of an image of the array 10 collected by the system of FIG. 1 is shown in FIG. 2. Some of the assay sites 15a comprise spots representative of a reaction, e.g. a reaction indicative of a presence of an analyte, some of the assay sites 15b are indicative of no reaction, which may be indicative of lack of an analyte, and some of the assay sites 15c are empty. The spots are generally associated with a change in opacity or colour or the like that is representative of the extent or degree of reaction. Importantly, the present inventors have identified situations in which different assay sites 15a, 15a′ of the array 10 are used to carry out a reactions having a different reaction strength. The different reaction strength could be associated with different assays, i.e. different types of assay are carried out in different assay sites 15 and at least one or each of the assays may be associated with a different reaction strength to at least one other of the assays. In another example, the reaction strength could be sample dependent or vary from assay site to assay site.

The analysis of the reaction in each assay site 15 determines one or more metric for the reaction in the assay site 15 and applies logic, e.g. a comparison to a threshold, that depends on one or more parameters to yield a measure of reaction state (e.g. reacted or not, i.e. a qualitative determination, or a value for extent or degree of reaction or activity, i.e. a quantitative determination, or an indication of active or not active or the like). This could be done by selecting a threshold for all of the reactions, e.g. assays and samples, for all of the assay sites. However, the present inventors have noted that the dependency of attributes of each analysis on a given metric can vary differently and significantly for different assays.

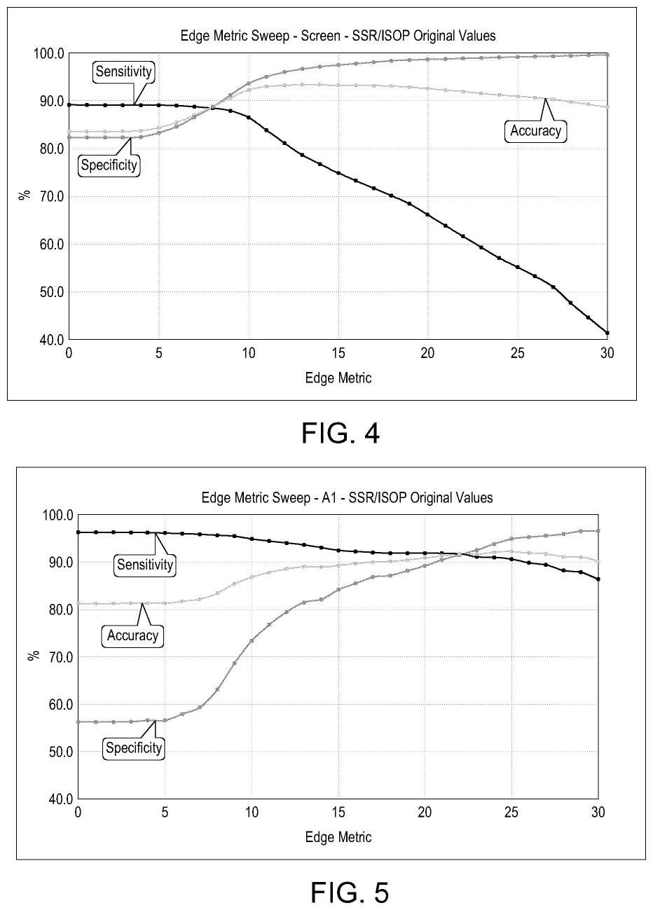

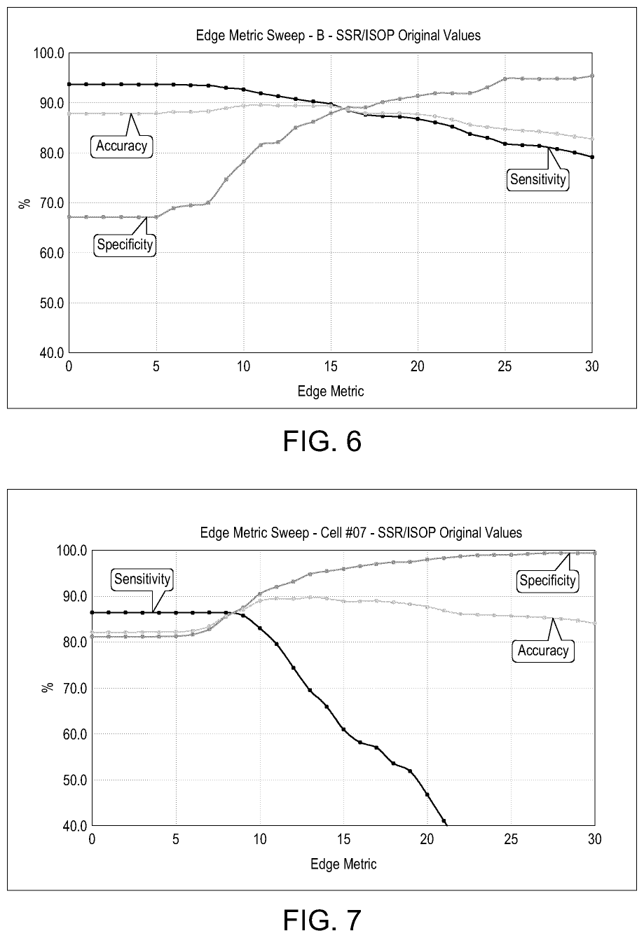

This can be seen from FIGS. 3 to 7, which each show the variation of different attributes (in this case accuracy, specificity, and sensitivity) with an exemplary metric (edge metric in this case) for different assays. As such, in these examples, if a common edge metric threshold is set for the assay shown in FIG. 3, then an edge metric threshold of 20 or above may be desirable. However, for the assay shown in FIG. 4, an edge metric threshold of 20 or above may result in an undesirably low sensitivity for that assay and an edge metric threshold of between 5 and 10 may be more preferable. It can be seen from each of the assays shown in FIGS. 3 to 7, that different threshold values of the metric may be better for different assays. Having identified these issues, the assay analysis system 5 is specifically configured to allow enhanced analysis of arrays that are used to perform different assays in different assay sites 15 of the same array 10.

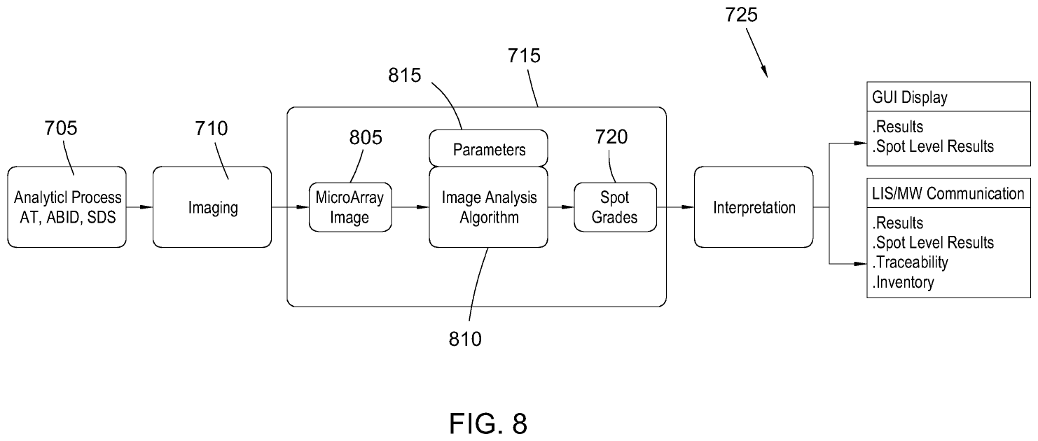

An overview of a computerized method of analysing the array 10 to determine an extent or degree of reaction in the assay sites 15 of the array 10 is shown in FIG. 8. Although the method can be performed by the processing system 25 shown in FIG. 1, one or more or each of the steps could be performed by the remote processing system 50 or different steps could be performed by the processing system 25 and the remote processing system 50 such that the processing is effectively distributed between them.

Step 705 indicates the performance of assays in each of a plurality of assay sites 15 of the microarray 10. The performance of individual assays is known in the art, although in the present case different assays are performed in at least some of the assay sites 15 of the microarray 10, and captured in an image 805 collected by the assay analysis system 5 in step 710.

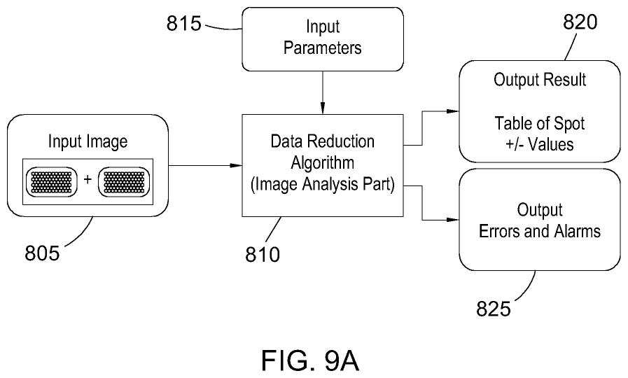

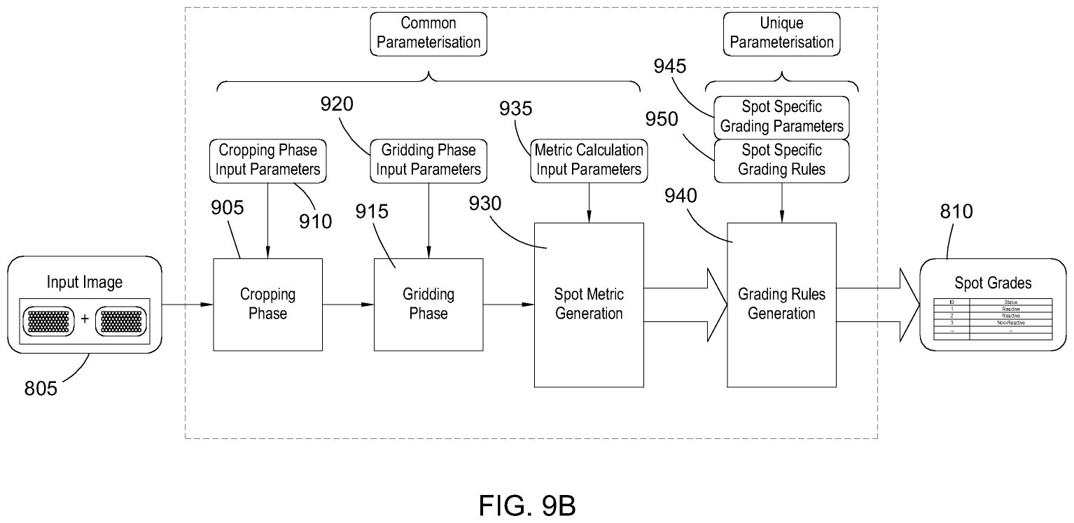

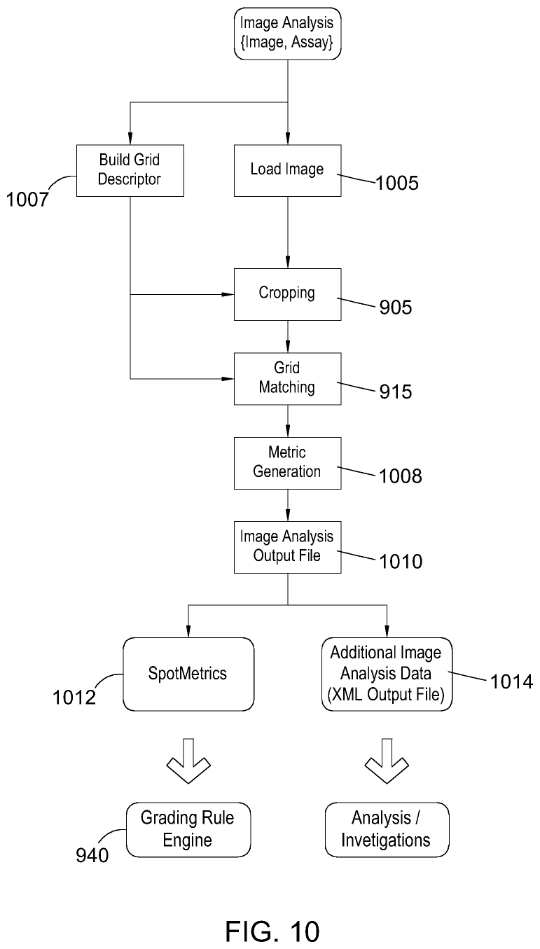

Algorithm 810 provides image analysis as part of the assay analysis, and is shown in further detail in FIGS. 9A, 9B and 10.

The processing system 25 and/or the remote processing system 50 receives the image 805 of the array 10 collected by the camera 17 (see FIG. 1). The processing system 25 and/or the remote processing system 50 implements the algorithm 810 shown in FIGS. 8 and 9A that processes the image 805 to determine metrics indicative of an extent or degree of reaction in each assay site 15. The algorithm 810 uses various parameters 815 to determine metrics indicative of the extent or degree of reaction for each assay site 15.

The parameters 815 used to determine the metrics are global and apply regardless of the assay being performed. However, the analysis of the metrics by the grading engine uses parameters that are specific and non-generic, i.e. different parameters can be used for different assay sites 15 or groups or subsets of assay sites 15 of the same array 10. For example, different assay types can be associated with different parameters. As the array 10 is used to perform different assays that are included in the image 805, the parameters for at least one of the assay sites 15 will be different to those for at least one other assay site 15 in the same array 10. The parameters for each assay type are generally predetermined and the appropriate predetermined parameters are selected for each different assay site depending on the type of assay being performed at that site. The assay type being performed in each different assay site and/or the parameters associated with that assay type is generally provided in a configuration file. For completeness, the provision of the parameters is not essentially limited to this and in other possible examples the assay type being performed in each different print area can be determined from user input received from one of the input devices such as a keyboard or a reader for an identifier provided on or with the array such as a QR code, RFID tag, holographic tag, barcode, or the like. For example, the assay type for each assay site 15 can be input by a user using the keyboard or the identifier for an array can be read by the reader, and pre-stored data for that assay type can be retrieved from a database, look up table or other form of data file that could be stored on the data storage 30 and/or in the remote data storage 55, and/or the like. Additionally or alternatively, the parameters can vary by sample type, sample size, by individual assay site position in the array 10, and/or by other factors that affect the reaction strength or extent or degree of response to the reaction. In this case, it will be appreciated that the parameters for each assay site 15 or group of assay sites 15 can be determined in a similar way to that described above.

The algorithm 810 is configured to analyse the image, identify the regions of the image associated with different assay sites 15, determine one or more metrics for the regions of the image associated with the different assay sites 15, and determine an extent or degree of reaction (such as reactive or unreactive or a value for extent or degree of reaction or activity) for the different assay sites 15 based on the metrics and the parameters 815.

As shown in FIGS. 9A, 9B and 10, the output from the algorithm 810 can include metrics 820 indicative of the extent or degree of reaction for one or more or each of the assay sites 15. The metrics for the assay sites 15 are processed by the grading engine to determine an extent or degree of reaction (e.g. spot grades) 720, which could be a simple indication of a reaction or no reaction, or could be more nuanced, e.g. a value giving an extent or degree of the reaction in the assay site. The determined spot grades can then be presented for interpretation by a user 725. The output could also include one or more errors and/or alarms 825, e.g. when the algorithm has failed to determine an extent or degree of reaction for a given assay site 15 or some other error in the process has occurred.

A more detailed overview of the process shown in FIGS. 8 and 9A is shown in FIG. 9B. As shown in FIG. 9B, the process has as an input the image 805 of the array 10 collected by the camera 17.

The input image 805 is cropped 905 so that the image 805 is tightly cropped to the area of the image 805 containing the assay sites 15 (and any other features used by the process such as control assay sites/spots) based on cropping parameters 910 that control the cropping process 905. Micro-arrays 10 can be provided in standardized sizes, dimensions and layouts. The cropping process is intended to reduce the area of the original image 805 to the area of the image associated with the array of assay sites 15 and any other features used by the process, such as control assay sites 15/spots. Cropping processes in general are known and any suitable cropping process could be used. An example of a suitable cropping process comprises identifying corner control spots and using these to determine an array area in the image where the array of assay sites 15 is located. The area is defined based on the position of the four control spots and a safe margin necessary to take the spot placement tolerance into account. The image can then be cropped around the array area.

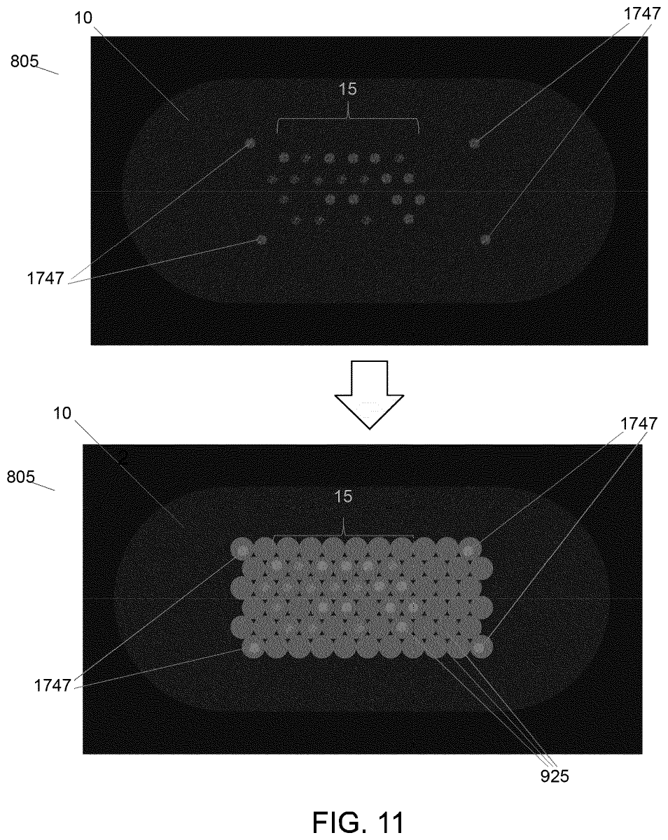

The cropped image output from the cropping stage 905 is subjected to gridding 915 that divides the cropped image into segments 925 (see FIG. 2), each segment 925 of the image containing a single assay site 15. The gridding 915 is performed based on gridding parameters 920 that control the gridding process 915. The gridding parameters 915 can be common parameters specific to the assay under test. The grid can be overlaid over the image. This comprises determining the most probable position of the grid based on the position of the corner spots. Segments 925 of a grid model corresponding to the corner control spots are known for the arrays 10, allowing the gird model to be applied to the image 805 using the determined positions of the corner control spots, as shown in FIG. 11.

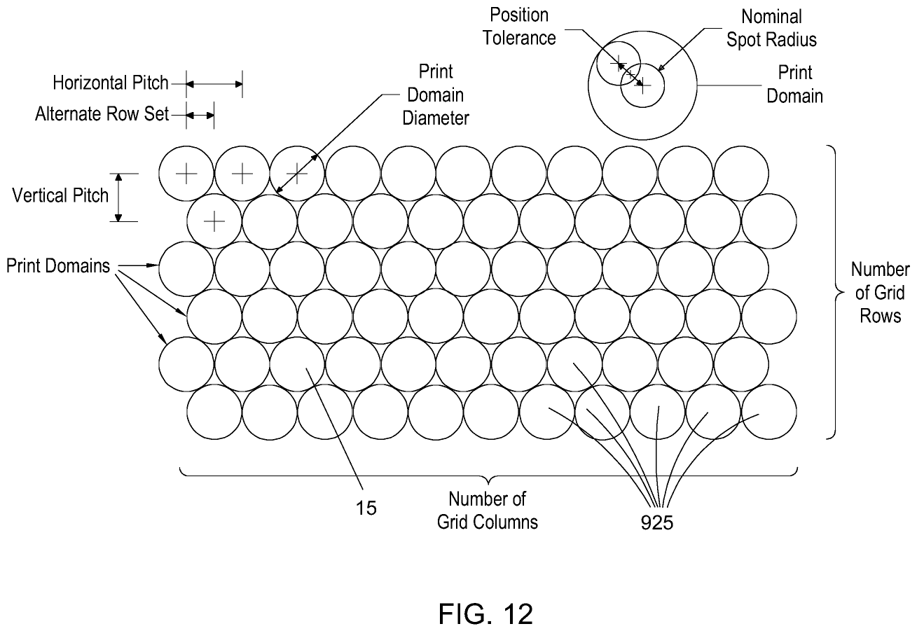

The gridding process 915 involves building the model of the array of assay sites 15 in the array 10 based on a geometric definition of a layout of the array of assay sites and on a position tolerance of the spot in the assay site 15. The grid is shown in FIG. 12 and organised in rows and columns of segments 925, each segment 925 encompassing a single assay site 15. A print domain corresponding to a single assay site 15 contains a single spot and a spot can't be in contact with a neighbouring spot. The result is a grid organised in rows and columns of print domains corresponding to the segments 925 of the grid. The model of the grid (the grid descriptor) is stored in the data storage 30 and/or the remote data storage 55.

Once the segments 925 of the image have been generated, spot metrics 930 for a spot in a given assay site 15 can be generated based on metric generation parameters 935.

The spot metrics 930 can be any metric of the image associated with a spot that can be used to characterize the spot indicative of a reaction in a given assay site 15. For example, at least one of the spot metrics 930 of a given spot can comprise or be representative of pixel intensities for an area of the image representing that spot. A particular example of this is a spot metric 930 of a given spot that comprises or is representative of a mean value of the pixel intensities for at least part of the area of the image representing that spot of that reaction site and/or a difference between a mean value of the pixel intensities for the area of the image representing that spot and a mean value of background pixel intensities. A specific example of a suitable metric for each spot is standard deviation ratio (SSR), which is the difference between the mean value of the pixel intensities for at least part of the image representing the given spot and the mean value of the pixel intensities for at least part of the background, all divided by a measure of spread (e.g. standard deviation) of the background pixel intensities for at least the part of the background.

The spot metric calculations 930 can be designed such that the metric calculation is repeatable and generally the same regardless of the spot being analysed, e.g. by using average pixel intensity values, by using masks to select pixels from specified areas of spots and background, and/or the like. As such, the metric generation parameters 935 can also generally be generic or common between spots, regardless of assay type.

Once the spot metrics 930 have been determined for any spots of interest, then the spot metrics can be assessed using a grading engine 940 to determine the extent or degree of reaction for the respective reaction being performed in the respective assay site 15 containing the respective spot. The grading engine 940 applies spot specific (that are also assay specific and/or sample type specific and/or the like) grading parameters 945 and/or grading rules 950 to the spot metrics determined for a given spot in order to determine the extent or degree of reaction (e.g. spot grade) for that given spot/assay site 15. As noted above, as different spots/assay sites 15 are for different assays and/or sample types, then the grading parameters 945 and/or rules 950 applied by the grading engine 940 for one or more spots/assay sites 15 are different to those applied for some other spots/assay sites 15 in order to determine the extent or degree of reaction (spot grade) for each spot/assay site 15.

In this way, common or generic parameters are applied where appropriate, e.g. for one or more of the cropping 905, gridding 915 and/or calculation of spot metrics 930, which promotes efficient processing for those processes for which individual parameterization has less of an effect. In contrast, bespoke rules 950 and/or parameters 945 are determined and used by the grading engine 940 for different spots/assay sites 15 and/or different groups of spots/assay sites 15 on the same array 10, where using parameters specific to that assay, sample type or other factor can have a significant difference on the performance and quality of the assays being performed. However, it will be appreciated that, in other examples, one or more or each of the cropping 905, gridding 915 and/or calculation of spot metrics 930 can be carried out using assay site 15 specific (e.g. assay, spot position and/or sample type specific) parameters.

An alternative flowchart showing the method of FIG. 9B is shown in FIG. 10, which shows the input image 805 being provided and loaded 1005. Parameters in the form of a build grid descriptor 1007 are generated and used to crop 905 and grid 915 the input image 805. Metrics 1012 for each segment 925 of the grid are determined (step 1008) and output in an image analysis output file to the spot grading engine 940, which uses the metrics to grade the spot in each segment 925/well 15. The results of the image analysis are also output (step 1014). The output results in step 1014 could contain, for example, the determined metrics 1012 and any additional image analysis data, such as error indications, that might be useful or requested for further investigation.

Segments 925 of the grid model corresponding to the corner control spots 1747 are known for the arrays 10, allowing the gird model to be applied to the image 805 using the determined positions of the corner control spots 1747 and other spots, as shown in FIGS. 11 and 12.

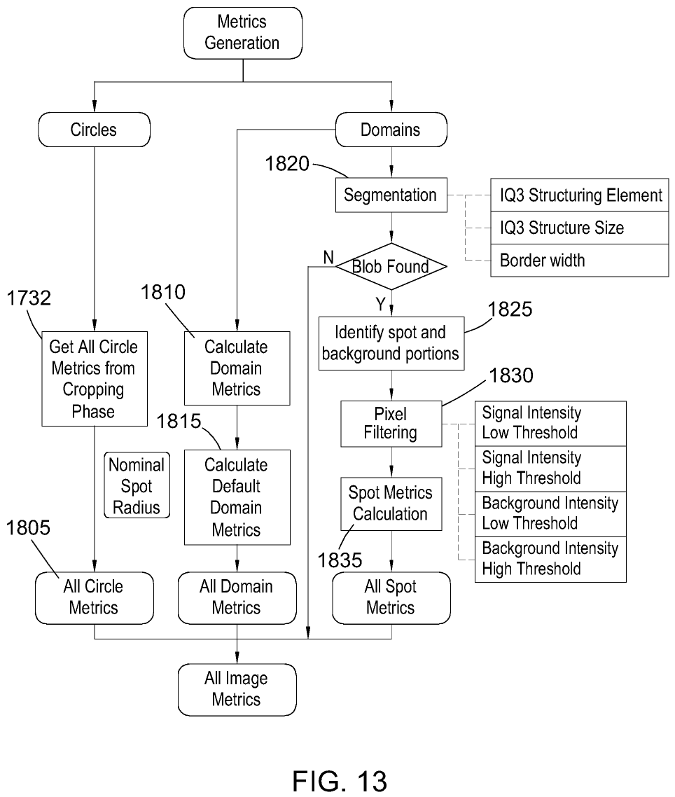

More detail of the generation of the metrics 930/1008 shown in FIGS. 9B and 10 is shown in FIG. 13. The metrics 930/1008 are determined from the image for each segment 925/print domain in the gridded image. The metrics 930/1008 for the segments (print domains) 925 are input into the grading rule engine 940, which uses the metrics to determine a grade or reactivity for each assay performed in the associated segment (print domain) 925.

As shown in FIG. 13, the metric generation process comprises calculating 1732 shape (e.g. circle) metrics 1805. The shape metrics are identified by using object recognition techniques to identify a shape (such as but not limited to a circle) indicative of a reaction spot in a segment and then determining metrics of the shape. The shape metrics can comprise, for example, one or more of: circle centre coordinates, circle radius, circle mean (i.e. mean pixel value of a circle area defined by the circle coordinates and radius), circle standard deviation (i.e. a standard deviation of pixel values in the circle area), circle background mean (i.e. a mean of the pixel values in an area of the background), circle background standard deviation (i.e. a standard deviation of the pixel values in an area of the background), circle SSR (a signal to standard deviation ratio for the circle area), any alternative metrics that are indicative of any of the above, and/or the like.

The metric generation process also comprises calculating domain metrics 1810 for each segment (print domain). The domain metrics can include metrics such as a domain mean (mean of the pixel values of an area defined by the entire segment coordinates and radius) and domain standard deviation (standard deviation of the pixel values of an area defined by the entire segment coordinates and radius), and/or the like. The domain metrics are calculated based on the position and size of individual segments of the grid.

In addition to calculating domain metrics 1810, the process also comprises calculating default domain metrics 1815 for each segment (print domain), the default domain being a circle of a default radius placed at the centre of the respective segment. Examples of default domain metrics include one or more or each of: a mean of the pixel intensities of the default domain, a standard deviation of the pixel intensities of the default domain, a mean of pixels of an area of background area of the default domain, a standard deviation of pixels of an area of background area of the default domain, and a signal to standard deviation ration (SSR) of the default domain, and/or the like.

The metric generation process further comprises calculating spot metrics. Spot metrics are extracted from each segment (print domain) by a segmentation method. The purpose of the segmentation step is to detect if an object (e.g. some form of activity or a “blob”, which may or may not be a spot caused by and/or indicative of the extent of the assay reaction) is present in the segment (print domain) under consideration and, if so, determine the position and size of the object. Corresponding metrics are calculated for both the object and for the background surrounding the object. If no object is found in a segment (print domain), no spot metrics are generated. If an object is detected, then the spot metrics are determined and subsequently used in the analysis performed by the grading rule engine to determine if the object is a spot and, if so, will determine the grade of the spot. Examples of possible spot metrics include: spot centre coordinates, spot radius, mean of the pixel values in an area defined by the spot mean and spot radius, standard deviation of the pixel values in an area defined by the spot mean and spot radius, mean and/or standard deviation of an area defined by the background, spot threshold, spot edge metric, spot isoperimetric coefficient, and/or signal to standard deviation ratio of the spot, and/or the like.

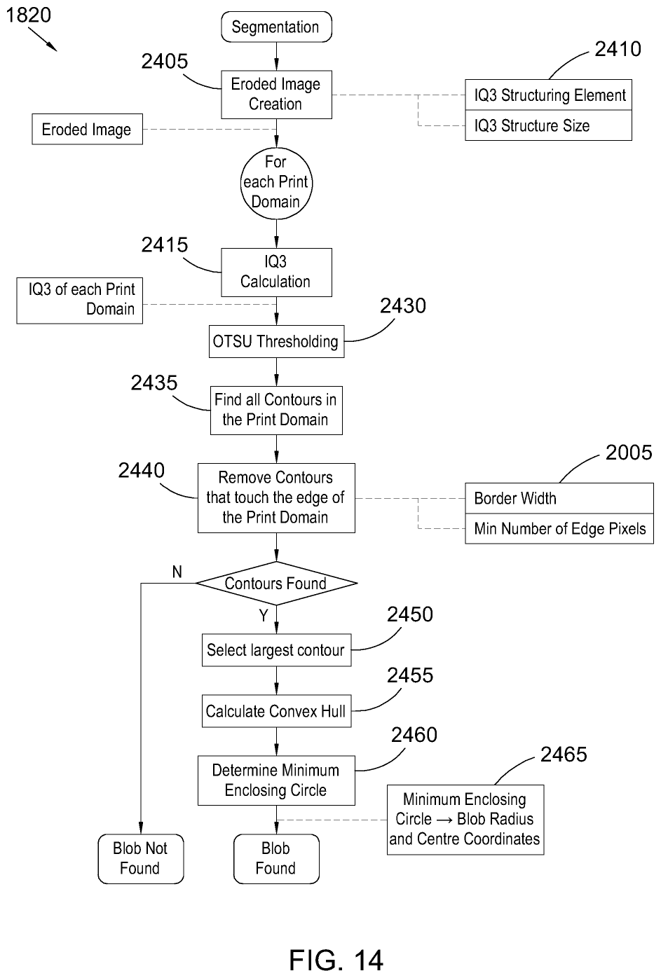

The determination of the spot metrics starts with a segmentation step 1820. The segmentation step 1820 is detailed in FIG. 14.

The segmentation process 2005 shown in FIG. 14 comprises creation of an eroded image 2405. This comprises applying a structuring element of a size and shape set by erosion parameters 2410, which include data setting a shape and size of the erosion element. The erosion element is a matrix that is placed on each image pixel, the value of the eroded pixel being the minimum input pixel found on the matrix. The erosion is used to determine an estimate of the local background. The structuring element can be chosen so that, after erosion, the centre of the segment reflects the local background.

For each segment 925, the segmentation process shown in FIG. 14 comprises determining a pixel intensity based metric of the segment 925. In this example, the metric for each segment 925 is an IQ3 metric 2415, which is calculated for the respective segments 925. The IQ3 metric for a segment 925 is the pixel intensity of the image at the centre of that segment 925.

In step 2430 of FIG. 14, thresholding is applied to segments 925. The thresholding converts colour or greyscale images into black and white images by applying a threshold on the pixel intensities, above which the pixel is determined to be white and below which is it determined to be black. The threshold can optionally be a dynamically determined threshold (i.e. to apply adaptive thresholding) or can be provided as a parameter.

In step 2435 of FIG. 14, the thresholded image output from step 2430 is analysed to determine any contours in the image. Various techniques can be used to identify contours and edges, and a suitable technique can be selected.

In step 2440 of FIG. 14, the contours found in step 2435 are checked on a segment 925 by segment 925 basis to see if they approach to within a border width threshold (which is provided as an input parameter 2005) of the border of the segment 925 and, if so, are removed. If more than one contour is left after this process, then the largest contour is selected for further processing, step 2450, and the remaining contours are discarded. If no contours are found for a given segment 925, then that segment 925 is labelled as “not segmented” or “blob not found” and no further segmentation processing is performed with respect to that segment 925.

For those segments 925 that have not already been labelled as “not segmented”, the convex hulls 2705 for the spots 2710 in those segments are determined in step 2455. The convex hull or convex envelope 2705 of a spot is the smallest possible convex shape that contains that spot 2710.

In step 2460, a minimum enclosing circle is determined for the respective segments. The minimum enclosing circle is the smallest possible circular shape that contains the convex hull. The minimum enclosing circle centre coordinates and radius are the output of the segmentation stage.

For each segment 925, if no blob is found in the segmentation process 1820 (i.e. it is unsegmented) then no spot metrics are determined for that segment 925. However, if a spot is determined to be present in a given segment 925, that segment 925 is partitioned into spot portions and background portions in step 1825.

The part of the image representing an assay site 15 associated with the segment 925 can be partitioned into portions representing the spot and portions representing a background. The portion representing the spot is used to calculate the average intensity of the spot, e.g. by taking a mean value of all of the pixel intensity values for pixels that are contained within the area of the portion representing the spot.

The portion representing the background is used to calculate the average intensity and standard deviation (or other measure of spread) of the background in the part of the segment that does not include the spot (i.e. the mean value and standard deviation of the pixel intensity values of pixels representative of the background).

For segments or spots that are determined to be segmented (i.e. a blob or some form of activity is present), then the process can proceed to determining the spot metrics needed for assessing the spot grade or extent or degree of reaction for each spot or assay site 15 that so far hasn't been deemed non-reactive or a technical error. Step 1830 of the process of FIG. 13 comprises pixel filtering the images to remove pixels that are atypical by being extremely high or low in intensity. Image artefacts typically have very high intensities and this process removes any remaining residues of these artefacts. Pixels with very low intensity may be indicative of noise. The pixel filtering applies high and low pixel intensity thresholds, which are parameters of the process, to both the spot pixel population and the background pixel population and removes those pixels below the low intensity threshold and above the high intensity threshold.

In step 1835, the spot metrics used in the spot grading/determination of the extent or degree of reaction are determined. Various metrics could be used. One metric is the signal to standard deviation ratio (SSR), which can be calculated for each spot/segment 925/assay site 15 using the following formula:

$$S S R = \frac{Mean ( Signal ) - Mean ( Background )}{StD ( Background )}$$

Where:
Mean(Signal) is the mean value of the pixels intensities in the filtered spot area;
Mean(Background) is the mean value of the pixels intensities in the filtered background area; and
StD(Background) is the Standard Deviation of the pixels intensities in the filtered background area

Other examples of metrics that could be used in addition or as alternatives to the SSR include IQ1, IQ2, IQ3 and IQ4, which are:

$$IQ 1 = abs ( \frac{Mean ( signal ) - Mean ( background )}{\begin{matrix}Background Factor * StD ( background ) + \\ SignalFactor * StD ( signal )\end{matrix}} ) - 1$$

$$I Q 2 = \frac{{A}_{signal}}{{A}_{spot}} ,$$

where A_{signal} is the area of the spot obtained using the spot radius, and Aₛₚₒₜ is either the area of the spot obtained from segmentation prior to pixel filtering (which is a measure of the number of pixels outside the Spot Radius) or the area of the spot within the spot radius excluding any pixels removed through filtering (which is a measure of the number of missing pixels inside the Spot Radius).
IQ3=the Pixel intensity of the resulting image at the centre of the segment 925
IQ4 is the ratio of the % of pixels at or above an intensity target to the % of pixels at camera saturation value (usually 255).

Other suitable metrics may be apparent to a skilled person from the teaching of the present disclosure. Any determined metrics are output to the grading rules engine 940. The grading rules engine takes the metrics, e.g. circle metrics, domain metrics and/or spot metrics for various of the assay sites, which are generally indicative of image properties, and uses these to determine spot grade or extent or degree of reactions, which are indicative of properties of the assay being carried out at the associated assay site.

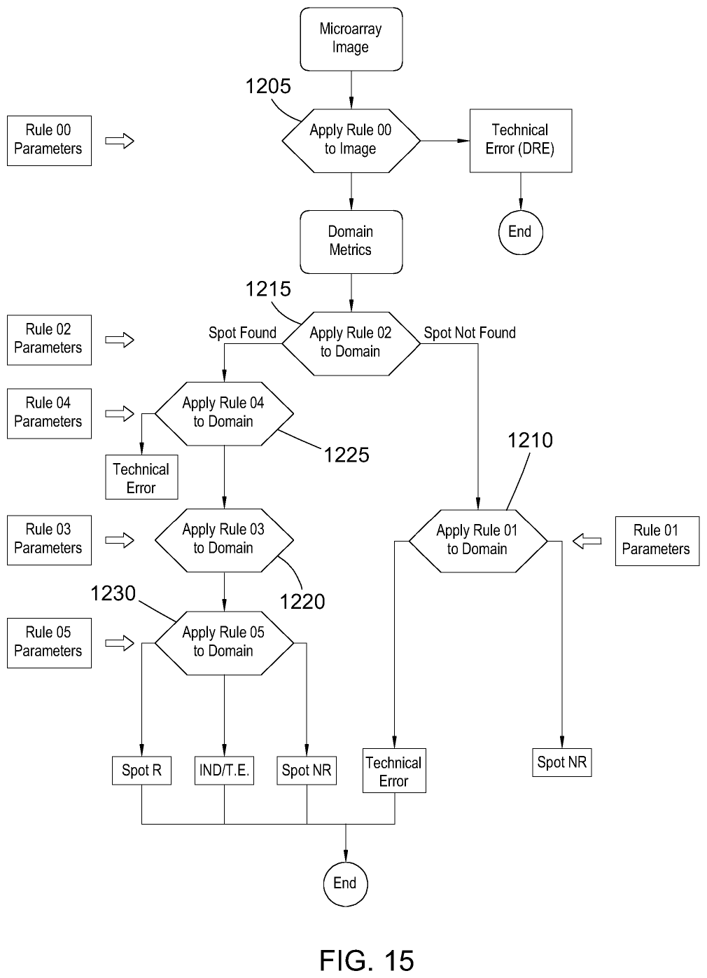

The grading rules engine 940 operates according to a logic that is outlined in FIG. 15. FIG. 15 is a flowchart giving an overview of rules used in the spot grading processes, i.e. the generation of spot/assay site 15 metrics 930 and outlines operation of the grading engine 940. The application of individual rules by the grading engine 940 is described in more detail below with respect to FIGS. 24 to 29.

In this example, the grading parameters 945 and rules 950 (as shown in FIG. 9B) used by the grading engine 940 to determine the spot grade or extent or degree of reaction can be either global or unique. Global parameters are parameters specific to a type of assay, whereas unique parameters are defined as a function of both the assay and the spot/assay site 15 position, i.e. the parameters are dependent on both the assay site 15/spot and on the assay.

The spot grading determination performed by the grading rules engine 940 considers the image 805 of the array of assay sites 15 in the array 10 (suitably cropped 905 and gridded 915 into segments) along with the metrics for each segment 925/assay site 15 and delivers the grading result for the corresponding spot/assay site 15/segment 925. In the specific example shown in FIG. 23, the grading engine 940 applies a set of six rules for each segment 925/assay site 15 of interest, namely:
Rule 00: A rule 1205 that identifies images not suitable for analysis;
Rule 01: A rule 1210 that identifies if the segment 925 is empty or contains an object;
Rule 02: A rule 1215 that identifies if the segment 925 contains a spot, e.g. by segmentation or circle search;
Rule 03: A rule 1220 that will grade a spot as reactive, non-reactive or either a technical error or the degree of reaction in of the spot/assay site 15 is indeterminate;
Rule 04: A rule 1225 that will detect bright objects; and
Rule 05: A rule 1230 that will confirm a result.

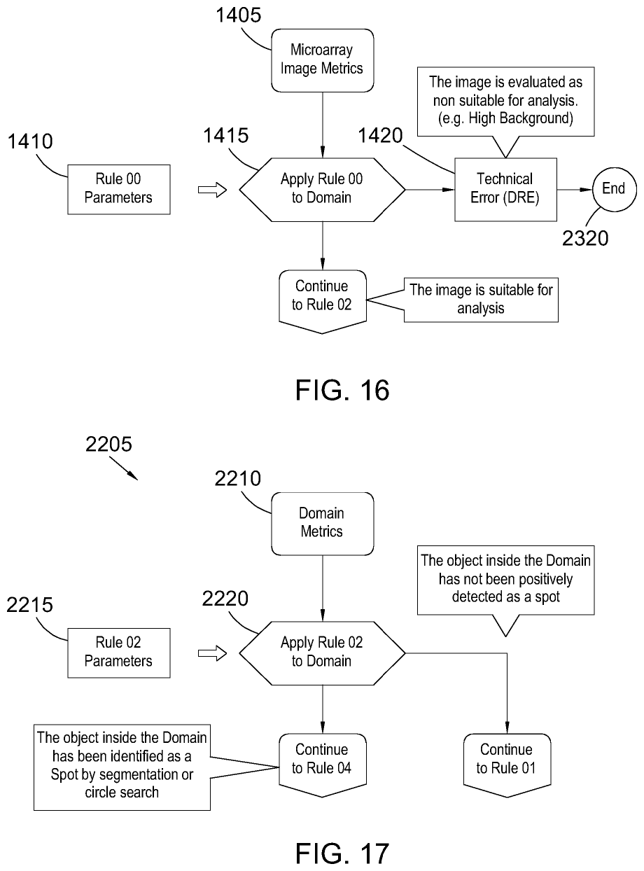

Although a useful set of rules 1205-1230 is provided above for grading the spots/reactions in assay sites 15 by the grading rules engine 940, in other examples only some but not all of the rules 1205-1230, or additional or alternative rules could be applied. The operation of the system will be described further with respect to FIGS. 16 to 21.

An example of flowchart of a process or rule for determining whether an image is suitable for analysis is shown in FIG. 16. The process or rule shown in FIG. 16 is operable to determine if an image of the array of assay sites 15 in the array 10 is suitable for analysis if the background of the image of the array 10 is sufficiently dark and uniform. The process uses 1405 metrics of the overall image, wherein the metrics of the overall image include, in this example, the mean of the pixel intensities for the background parts of the overall image and the standard deviation (or some other suitable measure of spread) of the pixel intensities for the background parts of the overall image. The rule uses global parameters 1410, including an image background mean maximum acceptable value (or threshold) and image standard deviation or spread maximum acceptable value (or threshold). The process comprises implementing a rule 1415 in which it is determined if either the mean of the pixel intensities for the background parts of the overall image exceeds the image background mean maximum acceptable value (or threshold) or the standard deviation (or some other suitable measure of spread) of the pixel intensities for the background parts of the overall image exceeds the image standard deviation or spread maximum acceptable value (or threshold) respectively. If so, then a technical error is flagged 1420 and the process ends 1425 for that image. Otherwise, it is determined that the image is suitable for analysis and the overall process implemented by the grading engine 940 continues to rule 1215, which is illustrated in FIG. 17.

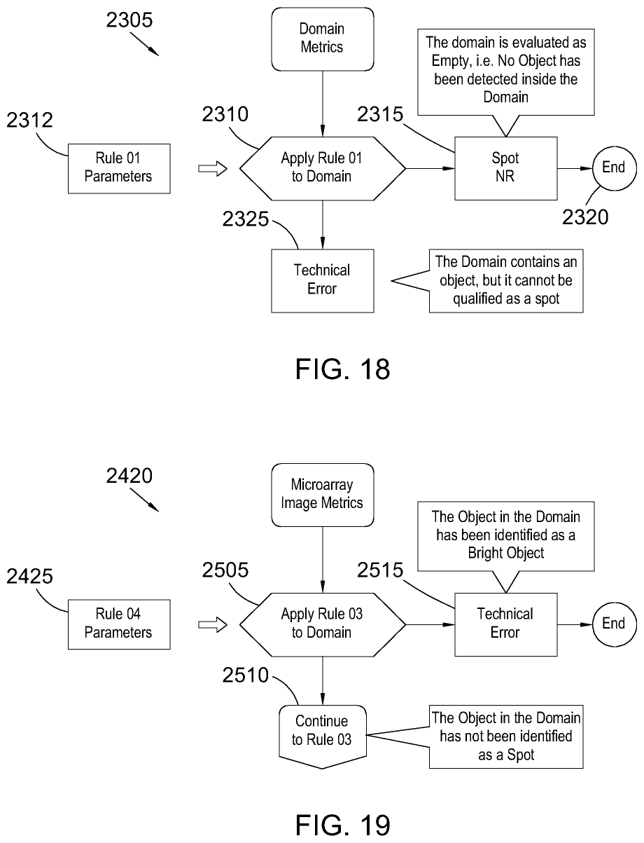

FIG. 17 is a schematic of a process 2205 for identifying if an object detected in a segment 925 is a spot indicative of a reaction. If the object can't be identified as a spot with a required certainty, then the process proceeds to the process 2305 of FIG. 18, which determines if the segment 925 is empty or not. The process 2205 of FIG. 17 determines an object in a given segment 925 is a spot indicative of a reaction if it satisfies certain conditions relating to the detectability, shape, size and position in the segment 925 of the spot.

The process includes receiving parameters 2215, such as maximum and minimum blob radius, maximum and minimum isoperimetric coefficient, maximum and minimum blob edge metric and the like. The parameters are unique for the individual assay site/assay being performed.

The process comprises applying rules 2220 to determine if metrics found in the segment support recognition of the object in a given segment are indicative of a reaction that is suitable for grading. If so, the graded reaction can be determined uniquely for the given segment.

The application of the rules 2220 utilise circle and domain metrics 2210, such as those generated during the metrics generation described above in relation to FIG. 13. The application of the rules 2220 can include, for example, determining if a circle has been found in a given segment 925. If a circle is found for the segment under analysis, then it is determined that a spot indicative of a reaction is present in that segment, and the radius and position of the circle are determined.

Additional or alternative rules can be used to determine the presence of a spot indicative of a reaction. For example, the application of the rules 2220 can also include, determining if an object in the form of a non-circular “blob” has been found based on SSR. If so, various metrics of the “blob” such as radius, edge metric, isoperimetric co-efficient, position and the like are determined. It is determined that a spot indicative of a reaction is present in the segment if all blob criteria are met, wherein the blob criteria comprise one or more of, for example, the blob radius is greater than or equal to the blob minimum radius and less than or equal to the blob maximum radius; the blob isoperimetric coefficient is greater than or equal to the blob minimum isoperimetric coefficient and less than or equal to the blob maximum isoperimetric coefficient; the blob edge metric is greater than or equal to the blob minimum edge metric and less than or equal to the blob maximum edge metric, and/or the like. If all of the blob criteria are met, then it is also determined that a spot has been detected.

If a circle is identified in the segment 925 or if all of the blob criteria are met, then the process 2420 shown in FIG. 19 is applied, which checks if the spot identified in the process 2205 can be identified as a bright object artefact rather than a spot indicative of a reaction.

If no circle was found and any of the blob criteria are not met, then the process 2305 of FIG. 18 is applied, which determines if a segment is empty or not.

If a segment 925 is determined to neither contain a circle nor meet the blob criteria, the process 2305 determines if the segment 925 contains an object or if it is empty. It does this by determining 2310 if the segment 925 is blank (i.e. the mean pixel value is below a certain level) or if it is uniformly filled with a level of grey that does not allow an object to be distinguished (i.e. no transitions or edges detected) and is consistent with the overall array image grey level. The determination 2310 comprises comparing certain metrics for the segment 925 (such as SSR value) to one or more thresholds that are set as parameters 2312 (such as a segment SSR limit parameter). This check can be specific for the printed area or assay in that segment 925.

For example, if an SSR value for the segment 925 is less than a segment SSR limit parameter, a mean pixel value for the segment 925 is less than a maximum mean pixel value parameter, a standard deviation (or other spread metric) for the segment 925 is less than a maximum standard deviation parameter, and an indication of a detection of an object to background noise less a measure of background noise is less than a maximum threshold parameter, then the segment 925 is determined to be empty 2315 and the process is ended 2320 for that segment 925. Otherwise, a technical error is determined 2325, that is, the domain contains an object but it cannot be said with sufficient certainty that the object qualifies as a spot indicative of a reaction.

As noted above in relation to the process shown in FIG. 17, if the process of FIG. 17 identifies that the object inside a given segment 925 can potentially be identified as a spot indicative of a reaction, the process comprises, as step 2420, removing bright object artefacts from each segment 925, which is shown in more detail in FIG. 19.

This bright object artefact removal step 2420, as shown in FIG. 19, comprises applying rules 2505 for determining if an object in a segment is a bright object anomaly by comparing a pixel intensity metric to one or more thresholds that are set as parameters 2425 and/or derived from other metrics. As examples, the rules 2505 may specify that an object identified in a segment 925 will be considered as a bright object anomaly if a signal mean value is above a detection threshold that is calculated from the IQ3 value or if the Signal Standard Deviation is above a value defined by a unique parameter, or the like.

The threshold is defined using a detection gradient and detection intercept that are provided as unique parameters 2425 for a given segment 925, and the detection threshold is defined as detection gradient for that segment 925 multiplied by the IQ3 value for that segment 925 plus the detection intercept for that segment 925. For example, the rules 2505 may specify that the object in a given segment 925 can be determined to be a bright object anomaly if any of the following criteria apply:
(1) The mean spot signal (e.g. pixel values) for the spot is greater than or equal to the detection threshold;
(2) The standard deviation or other measure of spread of the spot signal is greater than an associated threshold that is provided as a unique parameter 2425; or
(3) A mean of a circle signal is greater than or equal to the detection threshold;
(4) The standard deviation or other measure of spread of the circle signal is greater than an associated threshold that is provided as a unique parameter 2425.

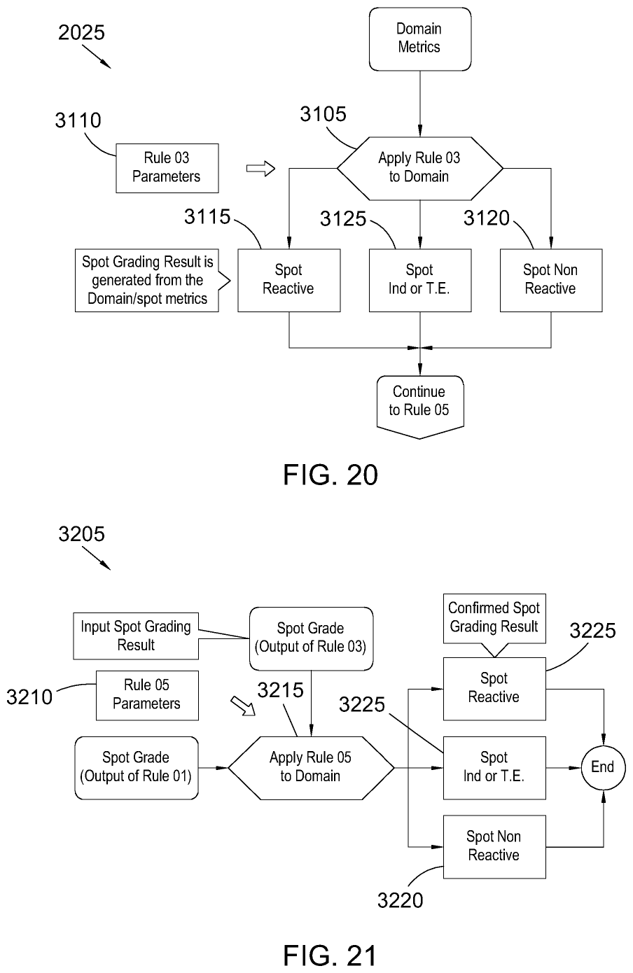

If none of these criteria are met, then the process, as step 2510, performs the process 2025 shown in FIG. 20, which applies rules to classify the spot. If any of the above criteria apply, then a bright object anomaly is detected and a technical error 2515 is recorded for that segment 925 (i.e. the detected object is likely an anomaly); and the process 2420 repeats for another segment 925 until all of the segments 925 in the image 10 have been analysed.

It will be appreciated that the bright object anomaly detection may use only one or some of the above criteria, and/or may use different criteria. In an alternative or additional step, the process may remove any pixels that have a pixel intensity above the detection threshold rather than rejecting the entire segment 925 on the grounds of a technical error.

As noted above, with respect to FIG. 19, if the process of FIG. 19 determines that the object is not a bight object artefact, then the process 2025 shown in FIG. 20 is applied, which applies rules to classify the spot.

In process 2025 shown in FIG. 20, the spot grade/degree of reaction for each assay site 15/spot/segment 925 is determined from the metrics determined using the process shown in FIG. 13 and parameters that are assay and/or assay site 15 specific. The spot grading/determination of the degree of reaction is only carried out for those spots/segments 925 that have not been rejected by any of the preceding rules applied by the grading engine 940.

Determining a spot grade/degree of reaction based on SSR is provided as a beneficial example. It will be appreciated that the determination of spot grade/degree of reaction could additionally or alternatively be based on other metrics such as, but not limited to, IQ1, IQ2, IQ3, IQ4, and/or the like.

In the example, four thresholds are set, which are set as assay dependent and optionally also spot specific and/or sample type dependent parameters. The thresholds are, in increasing value, Tel, Tl, Th and Teh, The spot grade/degree of reaction for a given spot/assay site 15/segment 925 is determined based on the SSR according to the following table 1:

TABLE 1

| SSR Value Range | Spot Grade |
|---|---|
| >Teh | Technical Error |
| [Teh-Th] | Reactive |
| ]Th to Tl[ | Indeterminate |
| [Tl to Tel] | Non-Reactive |
| <Tel | Technical Error |

The process 2025 comprises applying rules 3105 for determining spot grade/degree of reaction for each spot/assay site 15/segment 925, as shown in FIG. 20. According to these rules 3105, the spot will be graded as reactive, non-reactive or indeterminate based on values of metrics that reflect its intensity, such as the SSR or delta (the delta is the difference between the mean spot or circle pixel intensity value and the mean background pixel intensity value).

In the specific example shown in Table 1, if the SSR value of a segment is below a lowest threshold Tel or above a highest threshold then a technical error is determined. If the SSR is above the lowest threshold Tel but below an upper non-reactive threshold Tl, then a non-reactive grading is determined. If the SSR is above the upper non-reactive threshold Tl but below a lower reactive threshold Th, then an indeterminate state is determined, which is indicative of it not being possible to determine the reactive and non-reactive state gradings with sufficient certainty. If the SSR is between lower and upper reactive thresholds Th and Teh, then a reactive grading is determined. Although the specific example given in Table 1 uses SSR as the metric, it will be appreciated that the same concept could also be applied to other metrics.

Beneficially, the rules 3105 may comprise logic to dynamically switch between the metrics used for determining spot value/degree of reaction, to select the most suitable metric, for example switching being based on the standard deviation or other spread metric of pixel intensities of the background. In the example, the logic uses the SSR as a default and switches to another metrics, e.g. the delta, if it is found that the SSR is unsuitable. The switch between SSR and delta is made based on a measure of spread in background values, for example by using background CV values which is 100× the standard deviation of the background divided by the mean of the background.

For example, in some cases, the background surrounding a spot is non-uniform, causing the background standard deviation to have values well in excess of the average. As a result, the SSR is strongly decreased, which can cause false non-reactive determination even when the spot is clearly indicative of a reaction. In this case (invalid SSR), an alternate spot reactivity evaluation using the delta is used and the background uniformity is evaluated using the CV instead of the standard deviation to take into account the average level of the background relative to the standard deviation.

Threshold parameters 3110 for the determination of spot grade/degree of reaction are all assay dependent, and optionally also dependent on the spot position and/or sample type, i.e. they vary for different assay sites 15 in the same array 10. The threshold parameters 3110 required include a maximum acceptable background CV value, reactive SSR value range, non-reactive SSR value range, reactive spot delta value range, non-reactive delta value range, and/or the like.

From the process described above in relation to FIG. 17 (rule 2), the spot may have been determined by segmentation or by circle detection. The threshold parameters 3110 that are applied may also depend on whether the spot is determined as a circle from the circle search or as a spot or “blob” via the segmentation process, with different threshold parameters being applied for each.

The application of the rules 3105 starts by determining if the background CV value is less than the maximum acceptable background CV value. If so, then the process proceed by using the SSR metric. If not, then the process proceeds using the delta metric, thereby providing the dynamic switching described above.

If proceeding using the SSR metric, it is determined if the SSR (either for the blob or the circle, depending on how the spot was determined) is within the reactive SSR value range (which is a threshold parameter) and if so determines that the spot/assay site 15/segment 925 is indicative of the associated assay being reactive 3115. If the SSR is not within the reactive SSR value range, then it is determined if the SSR is within the non-reactive SSR value range (which is also a threshold parameter) and if so then it is determined that the spot/assay site 15/segment 925 is indicative of being unreactive or not-reacted 3120. If the SSR for the spot is determined to be out with both the reactive SSR value range and non-reactive SSR value range, then it is determined that the spot/assay site 15/segment 925 is indeterminate or a technical error has arisen 3125.

If it is determined that the delta metric should be used, then a determination process similar to the above but using delta and threshold parameters 3110 (such as reactive delta value range and unreactive delta value range) associated with the delta instead of SSR and the threshold parameters 3110 associated with SSR are used.

Regardless of the determination made in the process 2025 shown in FIG. 20, a check step 3205, shown in FIG. 21, is made. The check step 3205 comprises applying rules 3215 to the determination of spot grade/degree of reaction determined from the spot qualification step 2025 shown in FIG. 17. The check step 3205 shown in FIG. 21 is an optional step and can be switched on or off by setting an appropriate check parameter value 3210. If the spot qualification step 2025 of FIG. 20 determines that the spot/assay site 15/segment 925 should be graded as “non-reactive” 3120 and the same spot/assay site 15/segment 925 is indicated as “empty” 2315 by the process in FIG. 18, then the rules 3215 confirm the spot/assay site 15/segment 925 as non-reactive 3220. However, if the spot qualification step 2025 determines that the spot/assay site 15/segment 925 should be graded as “non-reactive” 3120 and the same spot/assay site 15/segment 925 is not indicated as “empty” 2315 by the process in FIG. 26, then a “technical error” 3225 is output for the spot/assay site 15/segment 925 instead of “non-reactive”. If the spot qualification step 2025 determines that the spot/assay site 15/segment 925 should be graded as “reactive” 3115, then this is maintained by the rules 3215, then the “reactive” determination is output 3230.

Beneficially, at least the threshold parameters used in the spot qualification process 2025 described above in relation to FIG. 20 are separately variable depending on factors such as one or more of: the assay being performed, the specific location of the assay site 15 on the assay 10, the type of sample being analysed, and/or the like. In this way, the effectiveness of the assay determination can be improved by selecting better thresholds for any given assay, assay site 15 and/or sample type, regardless of what other (different) assays are being performed on the same array 10. That is, less compromise in the setting of threshold parameters is needed to account for performing different assays in different assay sites 15 on the same array 10.

Various steps prior to the spot qualification are carried out, which need not be essential, but individually or in combination, may improve the accuracy or efficiency of the assay result determination.

Although a specific example is described above, it will be appreciated that this is provided to give the skilled person one possible way to put the invention into practice, but variations to the methods and apparatus described above are possible within the scope of the claims.

For example, various metrics such as SSR, delta, IQ3 and the like are used, but it will be appreciated that alternative metrics could be used. Furthermore, although various techniques for detecting spots in the assay sites 15 are described above, such as edge detection, thresholding, and shape, e.g. circle identification, and the like, it will be appreciated that alternative techniques could be used.

Digital cameras 17 are advantageously used to collect the images of the assays, but it will be appreciated that other sensor devices that are usable for determining metrics representative of a degree of reaction such as thermal cameras, ultra-violet or infra-red sensors and the like could be used.

Method steps of the invention can be performed by one or more programmable processors executing a computer program to perform functions of the invention by operating on input data and generating output. Method steps can also be performed by special purpose logic circuitry, e.g., an FPGA (field programmable gate array) or an ASIC (application-specific integrated circuit) or other customised circuitry. Processors suitable for the execution of a computer program include CPUs and microprocessors, and any one or more processors. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, e.g. EPROM, EEPROM, solid state memory such as SSD and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

The method steps can be performed by a local processing device, on a remote processing device or certain steps can be performed on the local processing device and certain method steps can be performed on the remote device in a distributed processing arrangement.

To provide for interaction with a user, the invention can be implemented on a device having a screen, e.g., a CRT (cathode ray tube), plasma, LED (light emitting diode) or LCD (liquid crystal display) monitor, for displaying information to the user and an input device, e.g., a keyboard, touch screen, a mouse, a trackball, and the like by which the user can provide input to the computer. Other kinds of devices can be used, for example, feedback provided to the user can be any form of sensory feedback, e.g., visual feedback, auditory feedback, or tactile feedback; and input from the user can be received in any form, including acoustic, speech, or tactile input.

A suitable processing device for performing the method described above may comprise a mobile, fixed or network enabled device, comprising or configured to implement a controller or processing system. The device may be or comprise or be comprised in a mobile phone, smartphone, PDA, tablet computer, laptop computer, and/or the like. The controller or processing system may be implemented by a suitable program or application (app) running on the device. The device may comprise at least one processor, such as a central processing unit (CPU), maths co-processor (MCP), graphics processing unit (GPU), and/or the like. The at least one processor may be a single core or multicore processor. The device may comprise memory and/or other data storage, which may be implemented on DRAM (dynamic random access memory), SSD (solid state drive), HDD (hard disk drive) or other suitable magnetic, optical and/or electronic memory device. The at least one processor and/or the memory and/or data storage may be arranged locally, e.g. provided in a single device or in multiple devices in in communication at a single location or may be distributed over several local and/or remote devices. The device may comprise a communications module, e.g. a wireless and/or wired communications module. The communications module may be configured to communicate over a cellular communications network, Wi-Fi, Bluetooth, ZigBee, near field communications (NFC), IR, satellite communications, other internet enabling networks and/or the like. The communications module may be configured to communicate via Ethernet or other wired network or connections, via a telecommunications network such as a POTS, PSTN, DSL, ADSL, optical carrier line, and/or ISDN link or network and/or the like, via the cloud and/or via the internet, or other suitable data carrying network. The communications module may be configured to communicate via optical communications such as optical wireless communications (OWC), optical free space communications or Li-Fi or via optical fibres and/or the like. The device and/or the controller or the at least one processor or processing unit may be configured to communicate with the remote server or data store via the communications module. The controller or processing unit may comprise or be implemented using the at least one processor, the memory and/or other data storage and/or the communications module of the device.

1. A computer implemented method of analysing assays performed at respective assay sites of an array or microarray that comprises a plurality of assay sites, the method comprising:
receiving at least one image, the at least one image collectively or individually imaging the plurality of assay sites of the array or microarray, processing the at least one image to determine at least one metric representative of the degree of reaction at that assay site;
for each of the assay sites, determining one or more parameters for that assay site, wherein the one or more parameters for at least one of the assay sites of the array or microarray are different from the one or more parameters for at least one other of the assay sites of the array or microarray; and
for each of the assay sites, determining an extent of the reaction at that assay site from the at least one metric for that assay site and the one or more parameters for that assay site.

2. The method of claim 1, wherein:
the parameters comprise one or more criteria, thresholds or ranges;
each criterion, threshold or range is indicative of a different degree of reaction; and
the determining of the extent of the reaction at an assay site comprises determining whether or not the at least one metric for that site is above or below the one of more thresholds or within or out with the one or more ranges or does or doesn't meet at least one of the criteria to determine the degree of reaction.

3. The method of claim 1, wherein the parameters for a site depend on one or more of: an assay type performed at the site, a location of the site on the array or microarray, and/or a targeted assay type

4. The method of claim 1, wherein the at least one metric for a site comprises or is representative of pixel intensities for an area of the image representing a spot at that site, the spot being formed by reaction of an analyte and having a property indicative of the degree of reaction.

5. The method of claim 4, wherein the at least one metric for a site comprises signal to standard deviation ratio or a delta that is the difference between mean pixel intensity value for the spot and a mean background pixel intensity value.

6. The method of claim 1 comprising at least one of:
cropping the image around the array of sites; and/or
gridding the image into segments wherein each segment encloses a spot and/or assay site.

7. The method of claim 6, wherein at least one of: the cropping, gridding and/or the determining of the one or more metrics for the assay site uses common parameters that are shared with one or more or each or every other spot or site with the same assay type.

8. The method of claim 1, wherein the parameters for metrics for determining an extent of the reaction at the assay site are unique for that site.

9. The method of claim 4, wherein the method comprises detecting or identifying the parts of the images corresponding to the spots in the reaction sites comprising one or both of:
detecting or identifying shapes in the image that comprise circles of a diameter within a predefined interval; and/or
edge detection to determine the edges of the spots in the at least one image.

10. The method of claim 9, comprising filtering out identified spots having a measure of pixel intensity less than a threshold; and/or outside a predefined geometric area corresponding to the location of the assay sites on the array or microarray and positioned based on at least one other identified spot, control spot, or reference point on the array or microarray.

11. The method of claim 1, wherein the parameters used to determine the extent of the reaction at the assay site comprise at least one of: parameters received from a user input device and/or parameters retrieved from a data store corresponding to one or more identifiers associated with the array or microarray or one or more or each of the assay sites or the assays being performed therein.

12. The method of claim 11, wherein the identifiers are obtained by an input device comprising at least one of a user input device for receiving user input, a barcode reader, a QR code reader or other machine readable code reader, an RFID tag reader, and/or an infra-red signal reader.

13. The method of claim 1, wherein the determining of the extent of the reaction at the assay site comprises performing one or more logic tests to the at least one metric for that assay site and the one or more parameters for that assay site, where the result of the one or more logic tests is the extent of the reaction at the assay site comprising an indication of at least one of:
whether or not there has been a reaction at that assay site;
a degree of reaction at that assay site;
whether or not there is activity at that assay site;
a degree of activity at that assay site; and/or
whether or not an analyte is present at that assay site.

14. An assay analysis system for analyzing assays performed at respective assay sites of an array or microarray that comprises a plurality of assay sites, the assay analysis system comprising at least one processing device, data storage and a communications system for receiving images and outputting an indication of an extent of a reaction at an assay site; the processing device being configured to:
receive at least one image, the at least one image collectively or individually imaging the plurality of assay sites of the array or microarray, processing the at least one image to determine at least one metric representative of the degree of reaction at that assay site;
determine, for each of the assay sites, one or more parameters for that assay site, wherein the one or more parameters for at least one of the assay sites of the array or microarray are different from the one or more parameters for at least one other of the assay sites of the array or microarray; and
determine, for each of the assay sites, an extent of the reaction at that assay site from the at least one metric for that assay site and the one or more parameters for that assay site.

15. The assay analysis system of claim 14 comprising:
at least one imaging device configured or configurable to collect at least one image of the array or microarray and communicate the at least one image to the processing system; and
an output device configured to receive the an indication of an extent of a reaction at an assay site from the processing system and to output the an indication of an extent of a reaction at the assay site.

16. A computer program product comprising a non-transient computer usable medium having computer readable instructions embodied therein, the computer readable instructions being such that when implemented on a processing system, cause the processing system to perform an assay analysis for analyzing assays performed at respective assay sites of an array or microarray that comprises a plurality of assay sites that comprises:
receiving at least one image, the at least one image collectively or individually imaging the plurality of assay sites of the array or microarray, processing the at least one image to determine at least one metric representative of the degree of reaction at that assay site;
determining, for each of the assay sites, one or more parameters for that assay site, wherein the one or more parameters for at least one of the assay sites of the array or microarray are different from the one or more parameters for at least one other of the assay sites of the array or microarray; and
determining, for each of the assay sites, an extent of the reaction at that assay site from the at least one metric for that assay site and the one or more parameters for that assay site.
